(19) United States
(12) Patent Application Publication
(10) Pub. No.: US 2009/0191128 A1
(43) Pub. Date: Jul. 30, 2009

(54) CORE-SHELL NANOPARTICLES FOR THEARAPY AND IMAGING PURPOSES

(75) Inventors: Cornelis Reinder Ronda, Aachen (DE); Helga Hummel, Aachen (DE); Thomas Juestel, Witten (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (DE)

(21) Appl. No.: 12/307,786

(22) PCT Filed: Jul. 2, 2007
(86) PCT No.: PCT/IB07/52557

§ 371 Date: Jan. 7, 2009

(30) Foreign Application Priority Data
Jul. 10, 2006 (EP) 06116923.1

(51) Int. Cl. A61K 49/04; A61K 9/14; A61K 49/18

(52) U.S. Cl. 424/9.4; 424/490; 424/9.3; 977/773

(57) ABSTRACT

The invention relates to nanoparticles coated with an inorganic nanoscale material herein referred to as core-shell nanoparticle, in which the core material has a wider band gap than the shell material. The described core-shell nanoparticles are very suitable for the application in photodynamic therapy due to their enhanced energy transfer ability.

The invention relates to nanoparticles for therapy and imaging purposes, especially for the use in photodynamic therapy.

Photodynamic therapy (PDT) is based on the discovery that certain compounds can kill single-cell organisms on exposure to a particular type of light. It has been shown in the past that PDT is also able to destroy cancer cells by using a fixed-frequency laser light together with a photosensitizing agent.

In this type of cancer treatment a photosensitizing agent is applied into the vascular system where it is absorbed from cells all over the body. When compound-labeled cells are exposed to light, the chemical agent absorbs light. The absorbed energy can be transferred to oxygen molecules to generate an active form of oxygen that is able to destroy the treated tumor cell.

In more detail, a photon of light of an appropriate wavelength is absorbed by a photosensitizer molecule, raising it to a short-lived (singlet) excited state, the molecule can then undergo internal rearrangement to a longer lived (triplet) state which exchanges energy with molecular oxygen to produce highly reactive singlet oxygen (¹O₂). The photosensitizer then returns to the ground state where it may undergo further activation cycles.

Alternatively the activated photosensitizer reacts directly either with the substrate, such as the cell membrane or a molecule, transferring a hydrogen atom to form radicals. The radicals interact with oxygen to produce oxygenated products such as superoxide, hydrogen peroxide, hydroxyl, hydroxy-peroxyl or other oxygen radicals.

There are three known mechanisms by which PDT mediates tumor cell destruction. In the first case, a reactive oxygen species that is generated after the absorption of light energy, can kill tumor cells directly. PDT also damages the tumor-associated vasculature, leading to tumor infarction. Finally, PDT can activate immune response against tumor cells. These three mechanisms can influence each other and their combination is required for longer-term tumor control.

Examples of known photodynamic therapy drugs that are commercially available are porphyrins, purpurins, Phtalocyanines, texafrins, chlorins. These compositions are disclosed in B. C. Wilson, Can. J. Gastroenterol Vol 16 No 6, June 2002, page 393-396.

It is known from R. Bakalova et al., Nano Lett. (Communication), 2004, 4(9); p1567, that semiconducting nanoparticles (quantum dots) like CdSe facilitate the excitation of common PDT photosensitizers. It is also described that CdSe can generate singlet oxygen without mediating photosensitizer, but with significantly reduced quantum yield of generated singlet oxygen.

US 2003/0017264 A1 describes the production of semiconductor core-shell nanoparticles with good chemical, photochemical, and photophysical properties via the use of a reaction additive. The intended improvement of luminescence is achieved by the shell of these particles being more electronically insulating to the core excitation.

From US 2004/0033345 A1 it is known to make stable and water soluble metal or semiconductor core-shell nanoparticles by coating with a hydrophobic ligand which can be further encapsulated in a micelle structure. In an aqueous medium, the micelle comprises a hydrophilic shell and a hydrophobic core. The coated nanoparticles show an increased photoluminescence quantum yield and can be used in biological applications, such as fluorescent marking for determination and observation of the progeny of target cells.

The use of single luminescent semiconductor particles such as CdSe (quantum dots) show low quantum yields of generated singlet oxygen. Additionally, it does not allow absorbtion and emission properties to be optimised individually.

The core-shell nanoparticles of prior art show effective luminescence by keeping the exciton in the core. But they give insufficient energy transfer to photodynamic species, e.g. oxygen, in the environment due to the shielding effect of the shell material.

However, photosensitizers suitable for use in PDT have to provide an effective energy transfer to photodynamic molecules in order to be able to generate the reactive species.

Therefore, it is an object of the present invention to provide core-shell nano particles with increased energy transfer capability from the nano particles to nearby photodynamic molecules. It is another object of the present invention to provide nanoparticles for use in therapy or for medical, and/or imaging purpose, especially to provide nanoparticles suitable for use in photodynamic therapy (PDT).

We found that core-shell nanoparticles with a specific core/shell-material combination are very suitable for the application in photodynamic therapy, due to an enhanced ability of energy transfer.

The invention relates to nanoparticles coated with an inorganic nanoscale material herein referred to as core-shell nanoparticle, in which the core material has a wider band gap than the shell material.

One advantage of the present invention is that upon excitation the special material combination of the core-shell nanoparticle results in a field driven diffusion of the excitation to the surface of the particle. In this way, energy transfer between the luminescent core-shell nanoparticle and its surrounding tissue is optimised.

In more detail a favourable field gradient in a material combination of the invention is observed when the Fermi energy of the electron and the hole (exciton) in the core is larger than the Fermi energy of the electron and the hole in the shell. When the core-shell nanoparticles are excited, the absorbed energy results in the formation of excitons in the core material. Then the excitons diffuse field driven in addition to concentration driven into the shell material, i.e. the coating layer of the particle. The localised exciton can subsequently excite molecules next to the surface. In case of nearby oxygen this induces a very effective generation of singlet oxygen which can provoke cell death in the target tissue, e.g. in a photodynamic therapy application.

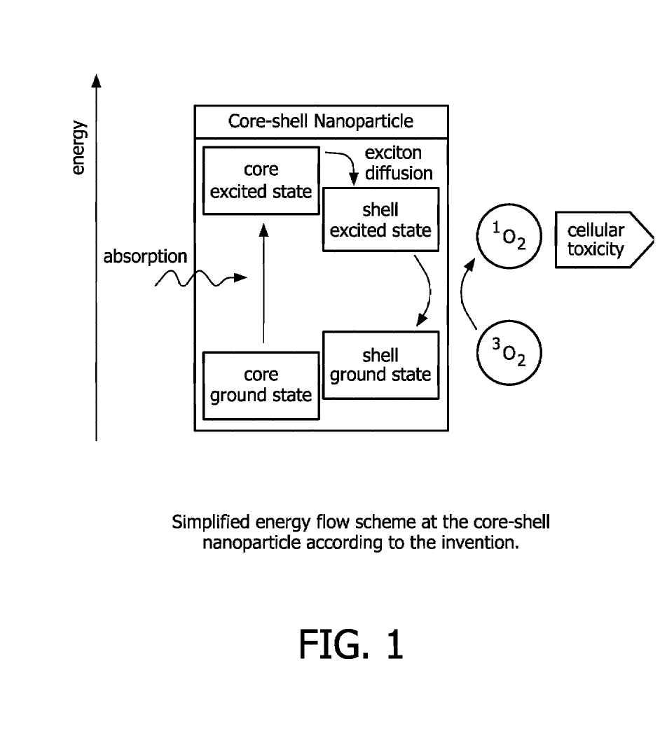

FIG. 1 shows a simplified energy flow scheme at the core-shell nanoparticle.

The present invention will be described with respect to particular embodiments but the invention is not limited thereto. Where the term “comprising” is used in the present description and claims it does not exclude other elements or steps. Where an indefinite or definite article is used when referring to a singular noun (e.g. “a” or “an”, “the”) this includes a plural of that noun unless something else is specifically stated.

Additionally the core-shell nanoparticles of the invention allow absorbtion and emission properties to be optimized individually.

Optical absorbtion can take place in the core when the shell is thin enough and it can be tuned by adjusting the core material and size.

Moreover the exciton energy to be transferred to the photodynamic species in the surrounding can be adjusted independent of the energy absorbed by tuning the material and thickness of the shell.

The invention allows the use of larger particles provided that the coating layer is only a few nm thick. In this way also luminescent particles based on up conversion schemes can be used in a much more effective manner. The shell has to be thin as this prevents too strong an absorption of the incident infrared light and hence inefficient up conversion. In addition, the excitons absorbed will, on their way to the shell, first diffuse to the surface of the core. This will increase the concentration of the excitons during their travel (in units 1/cm³) and hence the up conversion propability in the core.

Finally, up conversion schemes reduce day light exposure related problems with people undergoing PDT treatments.

The diameter of the core of the nanoscale particles according to the invention is in the range of 1 to 50 nm, preferably 5 to 50 nm, while the shell is 1 to 10 nm thick. Preferably the thickness of the shell is less than 10 nm, more preferably less than 5 nm.

Preferably the shell material does not react to a significant extent with body fluids.

The core and shell materials in the nanoparticles of the invention can be isostructural. Preferably the core and shell material have the same anion lattice. The anions of the materials are preferably selected from elements of the main groups V or VI of the periodic table.

Due to this selection of the core and shell materials the additional advantage is that the likelihood of local defects or shell cracking is minimized. Additionally the matching lattices allow the epitaxial growth of the shell material over the core material.

Preferably the core/shell-material combination is selected from the following pairs of compositions ZnS/CdS, GaN/InN, GaP/InP, Y₂O₃/In₂O₃, WO₃/MoO₃, ZrO₂/CeO₂. Generally elementary gold and SiO₂ are considered to be unfavourable shell materials.

Thus in one special embodiment of the invention the shell material is not elemtary Gold or SiO₂.

Upon excitation using a high-energy radiation source the core-shell nanoparticles of the present invention show formation of photons, having an energy of at least 1 eV. The emitted radiation can subsequently be transferred to molecules, e.g. oxygen, in the immediate environment. Especially for generation of singlet oxygen, e.g. in PDT in an aqueous medium, the energy transferred is preferably at least about 1 eV, more preferred 1.5 eV.

Examples for suitable excitation sources for the core-shell nanoparticles of the invention are high energy particles, X-rays, visible light, IR-, UV-radiation or X-ray. The preferred form of high energy radiation is X-ray radiation.

The core-shell nanoparticles of the invention can be used in all applications which require the effective energy transfer of the excitation to their environment. Especially the activated core shell nanoparticles of the invention can transfer their energy directly to small particles, molecules or ions in the surrounding tissue and lead to local generation of highly reactive species, e.g. singlet oxygen or nitrogen monoxide. The core-shell nanoparticles according to the invention can be used without an additional photosensitizer for effective singlet oxygen formation. They are stable in aqueous media, for example water, aqueous buffer systems, or mixtures of water with organic solvents. Additionally they show no or reduced photo-bleaching compared to known photosensitizers.

For sufficient energy transfer the distance between the nanoparticle and the photodynamic species to which the energy is transferred has to be rather small.

In another embodiment of the present invention the surface of the core-shell nanoparticle is provided with moieties, e.g. biomolecules to increase their biocompatibility and bioavailability. In this case the energy transferred to the shell can be transferred further by the biomolecules. Thus the photodynamic species, e.g. oxygen might be substantially farther from the shell.

In general it is desired that only specific target cells are affected by PDT whereas other cells, e.g. healthy cells that surround diseased tissue, are not affected. Therefore, optionally the nanoparticles according to the invention can be conjugated or linked to a targeting agent or targeting moieties like small molecules, antibodies, single chain antibody fragments, peptides, polypeptides, peptidomimetics, proteins, nucleic acids, lipids, saccharides etc. which are specific for the cells that should be treated by PDT.

Due to this functionalisation target specifity, the localisation of the photosensitizer in the target tissue and effectiveness of the photosensitizer can be optimised.

In another aspect of the present invention therapeutic and imaging purposes can be realised with the identical component.

The core-shell nanoparticles can be localized in vivo or in vitro prior to the real photodynamic therapy.

Core-shell nanoparticles having a suitable density can simultaneously be used as contrast agent in X-ray application.

The core-shell nanoparticles can provide an efficient conversion of X-ray photon energy into smaller energy packages, which can be used to generate the reactive species in cancerous tissue, simultaneous with X-ray therapy.

In another embodiment of the present invention the surface of the core-shell-nanoparticle is optionally provided with a magnetic resonance tomographie (MRT)-active ion complex or MRT active agent, such as Gd[DOTA] or Gd[DTPA] or Gd[DO3A] complexes. An overview on MRI contrast agents can be found in “Contrast Agents I, Magnetic Resonance Imaging”, 221 Topics in Current Chemistry, Volume Editor: W. Krause, Springer 2002 and in “Textbook of Contrast Media”, Edited by P. Dawson, D. O. Gosgrove, R. G. Grainger, ISIS Medical Media Ltd. Oxford 1999, Section II MR contrast agents, page 251-427. This enables localisation of the tissue to be treated with MRT, followed by treatments at the location needed, thus opimising the treatment and reducing side effects.

In another aspect of the invention the surface of the core-shell-nanoparticle is provided with nuclides which may be selected from ¹²³i, ⁶⁷Ga, ⁹⁹mTC, ¹⁸F and ¹¹C.

Due to this functionalisation of the particles they can also be used as contrast agent in nuclear medicine applications.

In a further embodiment of the invention the core-shell nanoparticles can be used for the preparation of medical, e.g. diagnostical and/or pharmaceutical, or therapeutic compositions, preferably for the preparation of a photodynamic therapy composition.

The compounds and medical or therapeutic compositions of the present invention may be administered locally or systemic, e.g. orally, parenterally, by inhalation spray, topically, rectally, nasally, buccally, vaginally, or via an implanted reservoir in dosage formulations containing conventional non toxic pharmaceutically acceptable carriers, adjuvants and vehicles. The term parental as used herein includes all kinds of injection or infusion techniques.

A medical or therapeutic treatment in accordance with the current invention can be used e.g. for the treatment of cancer, non-malignant tumors, auto-immune diseases, bacterial infections, acne bacteria or herpes, arteriosclerosis, arterial plaque, etc.. The therapeutic treatment method of this invention may be used in vivo or in vitro. In vitro treatment means e.g. treatment of cells that have been removed from animals or humans, such as kidney cells or liver cells, which are to be transplanted.

In another embodiment of the invention the core-shell nanoparticles and compositions can be applied in combination with other pharmaceuticals or chemical drugs.

Due to this combination therapy according to the invention anti-disease effects may be increased. In one example the photodynamic therapy composition is combined e.g. in form of a kit of parts, with a chemotherapeutic drug to increase anti-tumor effects.

The manufacturing method of the core-shell nanoparticles is in principle not critical and thus any conventional technique may be used. Several production techniques, as gas phase synthesis, solution phase synthesis, wet chemical synthesis are known. Gas phase synthesis may for example involve combustion flame, laser ablation, chemical vapor condensation, spray pyrolysis, electrospray and plasma spray. Examples for wet-chemical synthesis are sol-gel processing based on gelation, precipitation and hydrothermal treatment, colloidal synthesis or organometallic routes as hot injection techniques, high temperature thermolysis of organometallic precursors in the presence of stabilizing agents.

Synthesis of oxidic core shell-nanoparticles can be synthesised in aqueous solution starting from the corresponding acetates (Yttrium acetate, Indium acetate, etc.) Preferably in this case polyvinyl pyrrolidone or triphenylphosphinoxyd is used as surface stabilizing agents instead of thioglycerol.

The invention is illustrated by the following non-limiting examples.

### EXAMPLES

### Manufacture of CdS/ZnS Core-Shell Nanoparticles

0.1M of thioglycerol is added to 100 ml of a 0.1M solution of cadmium acetate in distilled water. Then 100 ml of a 0.1 M solution of Na₂S in ethanol are added dropwise under vigorous stirring. Subsequently the mixture is stirred for 2-3 h. After that the CdS particles are separated via centrifugation, washed with distilled water and dried by application of an IR lamp. The obtained CdS particles are redispersed in 0.1 M aqueous zinc acetate solution, followed by dropwise addition of 100 ml of 0.1M Na₂S solution in ethanol. Finally the mixture is stirred for 2-3 h and the particles were isolated via centrifugation.

1. Core-shell-nanoparticle in which the core material has a wider band gap than the shell material.

2. Core-shell-nanoparticle according to claim 1 in which the dark Fermi levels for both electron and holes in the core are larger than in the shell.

3. Core-shell-nanoparticle according to claim 1, in which the core material and the shell material are isostructural.

4. Core-shell-nanoparticle according to claim 1 with the core material and the shell material comprising the same anion lattice.

5. Core-shell-nanoparticle according to claim 1 in which the core/shell-material combination is selected from ZnS/CdS, GaN/InN, GaP/InP, Y2O3/In2O3, WO3/MoO3, ZrO2/CeO2.

6. Core-shell-nanoparticle according to claim 1 in which the surface of the core-shell nanoparticle is provided with a biocompatible moiety.

7. Core-shell-nanoparticle according to claim 1 conjugated to a target moiety.

8. Core-shell-nanoparticle according to claim 1 in which the surface of the core-shell-nanoparticle is provided with an MRT-active ion complex or MRT active agent.

9. Core-shell-nanoparticle according to claim 1 in which the surface is provided with nuclides.

10. Core-shell-nanoparticle according to claim 9 in which the nuclide is selected from 123I, 67Ga, 99mTC, 18F and 11C.

11. Core-shell-nanoparticle according to claim 1 for use as medical or therapeutical agent and/or for imaging purpose.

12. Use of core-shell-nanoparticles according to claim 1 for the preparation of a medical or therapeutic composition.

13. Use according to claim 12 of core-shell-nanoparticles for the preparation of a photodynamic therapy composition.

14. Use according to claim 12 of core-shell-nanoparticles in combination with other chemical drugs or pharmaceuticals.

15. Process for manufacture of nanoparticles according to claim 1 in which the shell material is grown epitaxially on the core material.
